(19) United States
(12) Patent Application Publication
(10) Pub. No.: US 2017/0105612 A1
(43) Pub. Date: Apr. 20, 2017

(54) VIDEO ENDOSCOPE WITH FLEXIBLE PRINTED CIRCUIT BOARD

(71) Applicant: OLYMPUS WINTER & IBE GMBH, Hamburg (DE)

(72) Inventors: Martin WIETERS, Hamburg (DE); Thorsten JUERGENS, Hamburg (DE); Sebastian JUNGBAUER, Hamburg (DE)

(73) Assignee: OLYMPUS WINTER & IBE GMBH, Hamburg (DE)

(21) Appl. No.: 15/393,969

(22) Filed: Dec. 29, 2016

Related U.S. Application Data
(63) Continuation of application No. PCT/EP2015/063671, filed on Jun. 18, 2015.

(30) Foreign Application Priority Data
Jul. 1, 2014 (DE) 102014212712.2

(51) Int. Cl. A61B 1/05; G02B 23/24; H05K 1/02; A61B 1/005

(52) U.S. Cl. CPC A61B 1/051; A61B 1/005; G02B 23/2484; H05K 1/028

(57) ABSTRACT

A flexible printed circuit board for use in a video endoscope is provided. The flexible printed circuit board including: a body extending longitudinally from a first end to a second end, the body having two end sections provided at the first end, the two end sections being spatially separate from each other relative to the longitudinal axis and each of the two end sections being configured to be connected to an imaging apparatus. A video endoscope having a flexible printed circuit board is also provided, where the first end of the flexible printed circuit board is arranged in the distal region of the video endoscope.

### CROSS-REFERENCE TO RELATED APPLICATION

The present application is a continuation of PCT/EP2015/063671 filed on Jun. 18, 2015, which is based upon and claims the benefit to DE 10 2014 212 712.2 filed on Jul. 1, 2014, the entire contents of each of which are incorporated herein by reference.

### BACKGROUND

Field The present application relates to a flexible printed circuit board for use in a video endoscope, as well as a corresponding video endoscope.

Prior Art

Flexible printed circuit boards are used in video endoscopes for transmitting video data and for supplying electrical energy to imaging apparatuses. Flexible printed circuit boards are connected electrically to an imaging apparatus in a distal region of the video endoscope. The traces of the flexible printed circuit board serve to conduct the data of the imaging apparatus to the proximal end of the endoscope where it can, for example, be forwarded for evaluation via plug-in connections and cables, for example. Correspondingly, power is supplied from the proximal end of the video endoscope via the flexible printed circuit boards to the distal end of the video endoscope.

With video endoscopes that, for example, have two or more imaging apparatuses, such as a 3-D video endoscope, a plurality of flexible printed circuit boards are normally used in order to be connected to the imaging apparatuses. This leads to a highly complex design and long assembly time.

With video endoscopes, it is important for the imaging apparatuses to be precisely aligned, such as being precisely aligned relative to each other, when a plurality of imaging apparatuses are used. In the production process, the imaging apparatuses may be shiftable and tiltable relative to each other in all three spatial axes. In addition, thermal alternating stress due to autoclaving can arise when using the video endoscope which can lead to mechanical stressing of the soldered connections of the imaging apparatuses to the respective printed circuit board.

An object is to enable a shorter endoscope assembly time with minimal design complexity and provide a corresponding flexible printed circuit board for the endoscopes. It is also the object to enable a precise alignment of the imaging apparatuses that are to be connected to a flexible printed circuit board.

### SUMMARY

Accordingly, a flexible printed circuit board for use in a video endoscope is provided, wherein the flexible printed circuit board extends longitudinally from a first end to a second end, wherein two end sections that are spatially separate from each other are provided at the first end to each be connected to an imaging apparatus.

With such flexible printed circuit board, it is possible to provide two imaging apparatuses in one video endoscope with little assembly effort, and install them quickly and precisely adjusted.

Each imaging apparatus can have a line of sight that may be arranged orthogonal to an image sensor plane of the respective imaging apparatus.

The end sections, and/or imaging apparatuses connected to the end sections, can be aligned such that an angle between 2° and 6° results between the respective lines of sight of the imaging apparatuses. A very true-to-nature three-dimensional image is possible through this measure.

The spatially separate end sections can be configured as arms.

The spatially separate end sections can oppose each other at a distance relative to a longitudinal axis of the flexible printed circuit board, or a main body of the flexible printed circuit board. The end sections can have at least one elastic bending region that is provided to compensate for the force acting on the end sections. This can allow the imaging apparatuses to be aligned well relative to each other.

The end sections can each have two bending regions that are arranged transverse, such as orthogonal, to each other. Efficient moving, or respectively tilting, of the imaging apparatuses can thereby be performed in all three spatial axes.

At least one end section can be configured as two C-shaped bodies that are not coplanar relative to each other in the state in which the flexible printed circuit board is installed in the video endoscope. Both end sections can be designed as C-shaped bodies that are not coplanar relative to each other in the state in which the flexible printed circuit board is installed in the video endoscope. This makes it possible to more easily align the two imaging apparatuses relative to each other, and the forces acting on the end sections can be compensated very easily, such that excessive forces cannot act on the soldered connections of the imaging apparatuses to the end sections.

A first C-shaped body can serve to compensate for longitudinal axial forces, and/or a second C-shaped body can serve to compensate for transverse axial forces.

A traction and/or torsion release point can be provided at a distance from the end sections that are spatially separate from each other at the first end of the flexible printed circuit board. At this point, it is possible to affix the first end of the flexible printed circuit board to a stable part of the video endoscope so that the connecting points to the imaging apparatuses are significantly relieved.

Also provided is a video endoscope with a flexible printed circuit board, wherein the first end of the flexible printed circuit board is arranged in the distal region of the video endoscope.

The traction and/or torsion release point can be spatially fixed in a tube of the video endoscope. This greatly relieves the stress at the connection of the imaging apparatuses to the flexible printed circuit board.

The imaging apparatuses can be each attached to an end section spatially separate from each other and can be spatially fixed in a tube of the video endoscope.

“Spatially affixing in a tube” can mean directly affixing in a tube. It can, however, also mean affixing to an element of the video endoscope that is arranged stationary relative to a tube of the video endoscope.

Further features will become apparent from the description of the embodiments together with the claims and the attached drawings. Embodiments can fulfill individual features or a combination of several features.

### BRIEF DESCRIPTION OF THE DRAWINGS

Exemplary embodiments are disclosed herein with reference to the drawings; without limiting the general inventive idea, wherein relative to all of the details not described in more detail in the text, reference is expressly made to the drawings, in which:

FIG. 1 illustrates a schematic view of a video endoscope with a flexible printed circuit board,

FIG. 2 illustrates a schematic plan view of a flexible printed circuit board in an unassembled state in another embodiment,

FIG. 3 illustrates a schematic plan view of a flexible printed circuit board according to FIG. 2 in a bent state as is the case in an installed state,

FIG. 4 illustrates a schematic side view along line of sight A from FIG. 3 of the flexible printed circuit board, and

FIG. 5 illustrates a schematic view along line of sight B from FIG. 3 of the flexible printed circuit board.

In the drawings, the same or similar types of elements and/or parts are provided with the same reference numbers so that a re-introduction is omitted.

### DETAILED DESCRIPTION

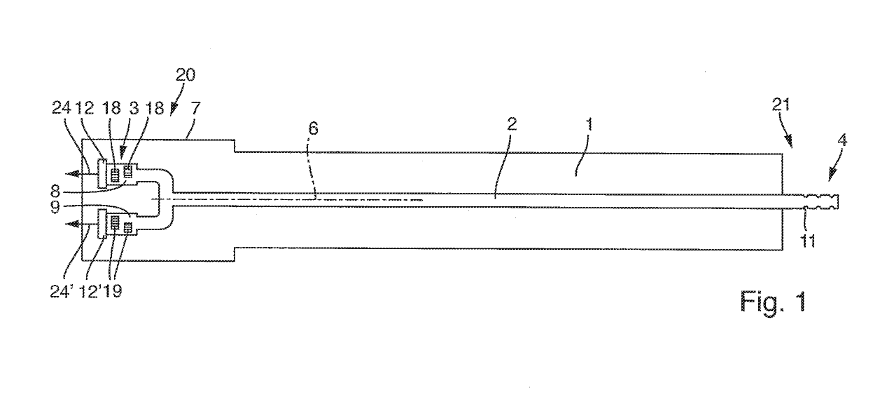

FIG. 1 illustrates a schematic plan view of a schematically depicted video endoscope 1 with a flexible printed circuit board 2 arranged therein. The printed circuit board 2 extends from a distal region 20 of the video endoscope 1 to a proximal region 21. Correspondingly, the flexible printed circuit board 2 is arranged elongated along the longitudinal axis 6 of a first end 3 to a second end 4, wherein the first end 3 is arranged in the distal region of the video endoscope 1.

The flexible printed circuit board 2 has corresponding traces which extend from the first end 3 to the second end 4.

A connecting piece 11 is provided at the second end 4 to connect the flexible printed circuit board 2, for example by a plug-in connection, to an evaluation electronics or an image recording apparatus.

The video endoscope 1 comprises, for example, a tube 7 in the distal region 20 which is stationary relative to the video endoscope 1. At the first end 3, the flexible printed circuit board is forked into first and second end sections 8 and 9 that are spatially separate from each other relative to the longitudinal axis 6.

One imaging apparatus 12 is attached to the first end section 8, and one imaging apparatus 12′ is attached to the second end section 9. These imaging apparatuses 12 and 12′ must be precisely positioned relative to each other, which is possible with the flexible printed circuit board. The positioning can be configured such that the lines of sight 24 and 24′ of the imaging apparatuses 12 and 12′, respectively, are at an angle of 2° to 6° relative to each other.

The imaging apparatuses can be connected by conductor pads, in particular, short printed circuit boards (not shown) to the flexible printed circuit board in the soldering regions 18, or respectively 19.

The two imaging apparatuses 12 and 12′ can be attached to a common flex board, or respectively the common flexible printed circuit board 2, by short, printed circuit boards, which may be flexible. The flexible printed circuit board 2 that can also be termed a flex board, has a type of branching at the first end 3, that is, i.e., at the tip of the endoscope 1 which is arranged in the distal region 20 of the endoscope 1, so that a corresponding connecting surface on which the soldering regions 18 and 19 are arranged, is provided for each imaging apparatus 12, 12′.

The video endoscope 1 is assembled particularly easily and quickly.

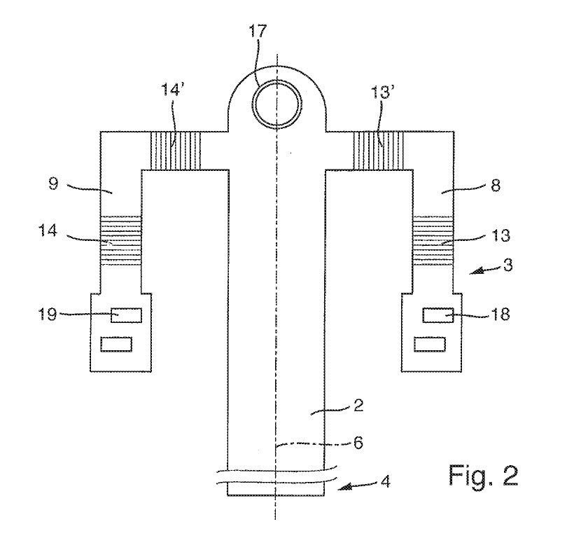

Another embodiment of the flexible printed circuit board 2 will now be described with regard to FIGS. 2 to 5. In this embodiment, the flexible printed circuit board 2 is provided at the first end 3 with two end sections 8 and 9 that are spatially separate from each other and first project transversely from the main body of the flexible printed circuit board 2 and then have an expanded section that projects transversely therefrom which, in the uninstalled state of the flexible printed circuit board in the embodiment according to FIG. 2, has an arm in each case that extends toward the second end 4.

In the embodiment according to FIG. 2, the two end sections 8, and 9 are designed as arms that first project orthogonal to the longitudinal axis 6 on the two sides of the flexible printed circuit board 2 and then continue further orthogonal thereto, i.e., parallel to the longitudinal axis 6.

Elastic bending regions 13, 13′ and 14, 14′ are provided, the end sections 8 and 9 of which can be correspondingly bent into C-shaped bodies.

At the end of the end sections 8 and 9, soldering regions 18 and 19 are provided to which the imagine apparatuses can be connected. The soldering regions shown here are, for example, provided with a type of window system.

Furthermore, FIG. 2 illustrates a traction and/or torsion relief point 17 which is provided as an opening, which can have a stable edge, by means of which a stable connection to e.g. a tube or another stationary element of a video endoscope is enabled by means of soldering or a bolt. This yields corresponding stability of the position of the flexible printed circuit board in the video endoscope.

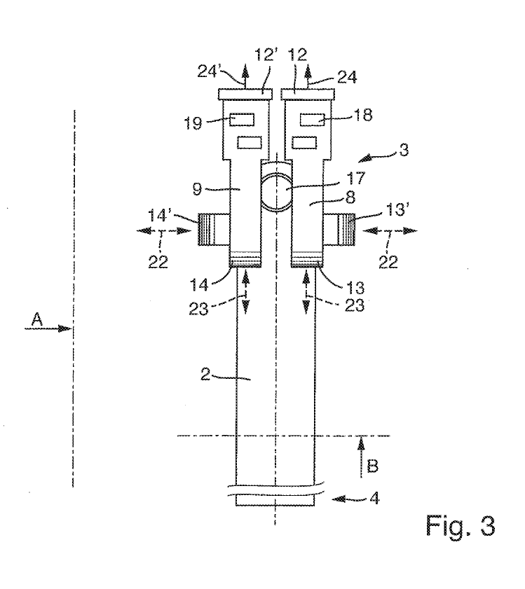

FIG. 3 illustrates the flexible printed circuit board 2 according to the embodiment of FIG. 2 in the bent state. By bending the flexible printed circuit board 2 at the elastic bending regions 13, 13′ and 14, 14′, a corresponding three-dimensional structure is produced which enables a very precise alignment of the imagine apparatuses 12, 12′ connected to the soldering regions 18 and 19. The imaging apparatuses 12, 12′ can be provided with lines of sight 24, 24′ that are at an angle of between 2° and 6° relative to each other.

In addition, this produces a release of force since the elastic bending regions can execute compensating movements 22 for lateral forces and compensating movements 23 for longitudinal forces.

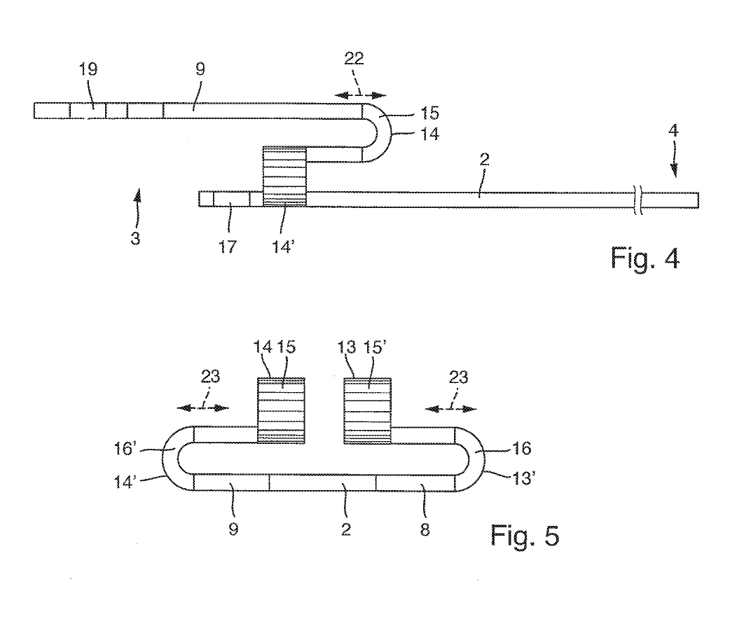

To better depict the three-dimensional design of the flexible printed circuit board 2 in an installed state in the video endoscope 1, FIGS. 4 and 5 illustrate side views. FIG. 4 illustrates aside view from side A, or respectively line of sight A as shown in FIG. 3, and FIG. 5 illustrates a view in direction B as shown in FIG. 3. The C-shaped bodies 15, 15′ and 14, 14′ are easily discernible that result from bending the elastic bending regions 13, 13′ and 14, 14′.

With such configuration, each arm can be individually moved in all spatial directions which is very efficient for precise assembly.

For stress relief, the main body of the flexible printed circuit board can be fixed in the video endoscope such that thermal stress cannot affect the two arms of the flexible printed circuit board. The fixation can be configured as fused-fit, force-fit or form-fit and can, for example, be achieved by adhering the main body of the flexible printed circuit board, or by screwed or soldered fixation to an eyelet as, for example, indicated by the traction and/or torsion relief point 17.

While there has been shown and described what is considered to be preferred embodiments, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### REFERENCE NUMBER LIST

1 Endoscope
2 Flexible printed circuit board
3 First end
4 Second end
6 Longitudinal axis
7 Tube
8 First end section
9 Second end section
11 Connecting piece
12, 12′ Imaging apparatus
13, 13′ Elastic bending region
14, 14′ Elastic bending region
15, 15′ C-shaped body
16, 16′ C-shaped body
17 Traction and/or torsion relief point
18, 19 Soldering region
20 Distal region
21 Proximal region
22 Compensating movement for lateral forces
23 Compensating movement for longitudinal forces
24, 24′ Line of sight

What is claimed is:

1. A flexible printed circuit board for use in a video endoscope, the flexible printed circuit board comprising:
a body extending longitudinally from a first end to a second end, the body having two end sections provided at the first end, the two end sections being spatially separate from each other relative to the longitudinal axis and each of the two end sections being configured to be connected to an imaging apparatus.

2. The flexible printed circuit board according to claim 1, wherein one or more of the two end sections and the imaging apparatuses connected to the two end sections are aligned such that an angle between 2° and 6° results between respective lines of sight of the imaging apparatuses.

3. The flexible printed circuit board according to claim 1, wherein the two end sections each have at least one elastic bending region provided to compensate for forces acting on the end sections.

4. The flexible printed circuit board according to claim 3, wherein the at least one elastic bending region comprises two bending regions arranged transverse to each other.

5. The flexible printed circuit board according to claim 4, wherein the two bending regions are arranged orthogonal to each other.

6. The flexible printed circuit board according to claim 4, wherein at least one end section is configured as first and second C-shaped bodies that are not coplanar relative to each other in a state in which the flexible printed circuit board is installed in the video endoscope.

7. The flexible printed circuit board according to claim 6, wherein at least one of the first and second C-shaped bodies serves to compensate for longitudinally axial forces.

8. The flexible printed circuit board according to claim 6, wherein a least one of the first and second C-shaped bodies serves to compensate for transversely axial forces.

9. The flexible printed circuit board according to claim 1, wherein the body further comprises a traction and/or torsion release point provided at a distance from the two end sections.

10. A video endoscope having a flexible printed circuit board according to claim 1, wherein the first end of the flexible printed circuit board is arranged in the distal region of the video endoscope.

11. The video endoscope according to claim 10, wherein the body further comprises a traction and/or torsion release point provided at a distance from the two end sections and the traction and/or torsion release point is spatially fixed in a tube of the video endoscope.

12. The video endoscope according to claim 10, further comprising the imaging apparatuses each attached to one of the two end sections to spatially separate the imaging apparatuses from each other and to spatially fix the imaging apparatuses in a tube of the video endoscope.
